(19) United States
(12) Patent Application Publication
(10) Pub. No.: US 2024/0038371 A1
(43) Pub. Date: Feb. 1, 2024

(54) SYSTEM AND METHOD FOR CREDENTIALING HEALTHCARE PROVIDERS

(71) Applicant: Law Office of Jerry Joseph, PLC, Washington, DC (US)

(72) Inventors: Simisola Isola, Bowie, MD (US); Keishauna Hayes, Saint Louis, MO (US)

(21) Appl. No.: 17/958,756

(22) Filed: Oct. 3, 2022

Related U.S. Application Data
(60) Provisional application No. 63/393,295, filed on Jul. 29, 2022.

(51) Int. Cl. G16H 40/20

(52) U.S. Cl. CPC G16H 40/20

(57) ABSTRACT

A computer implemented method for centralizing the credentialing process of healthcare providers including receiving credential information from each of a plurality of healthcare providers; storing the respective credential information of each healthcare provider; verifying the authenticity of the respective credential information of each healthcare provider; and communicating credential information to at least one of a plurality of healthcare providing entities.

### BACKGROUND OF THE INVENTION

### 1. Field of the Invention

The exemplary arrangements relate to systems and methods for credentialing healthcare providers such as nurses and others for contracted services and other employment.

### 2. Description of Related Art

There have been previous systems and methods that have been developed for credentialing healthcare providers. However, with these current systems and processes, once a healthcare provider's application has been submitted for a job by a recruiting agency, the recruiter will put the healthcare provider in contact with the interested healthcare providing entity's credentialing specialist, i.e., the onboarding specialist. If all requirements are satisfied, the individual healthcare provider then might get the job if the job has not already been filled. What makes this process even more confusing is that the healthcare provider is overloaded with emails from the credentialing specialist, often times asking the healthcare provider to email documents that have already been provided to the recruiting agency or the healthcare providing entity. The individual healthcare provider often receives numerous texts from different company phone numbers asking to upload these same documents, but when the number is called, it routes to an operator. These systems and processes may benefit from improvements.

Therefore, what is desired is a centralized system and method for credentialing individual healthcare providers through a secure network that enables quick dissemination of a healthcare provider's credential information, documentation and data to multiple interested healthcare providing entities, and which avoids risk of unwanted disclosure of the credential information.

### SUMMARY OF THE INVENTION

Exemplary arrangements provide a computer implemented method for centralizing the credentialing process of healthcare providers, comprising: receiving credential information from each of a plurality of healthcare providers; storing the respective credential information of each healthcare provider; verifying the authenticity of the respective credential information of each healthcare provider; and communicating credential information to at least one of a plurality of healthcare providing entities.

In alternative exemplary arrangements, the exemplary method further comprises receiving from a respective healthcare provider a request to release the health care provider's credential information to at least one healthcare provider selected healthcare providing entity.

In alternative exemplary arrangements, the exemplary method further comprises enabling a healthcare providing entity to establish qualifying criteria, wherein the qualifying criteria comprises specific credential information that the respective healthcare providing entity requires its healthcare providers to obtain.

In alternative exemplary arrangements, the exemplary method further comprises storing the qualifying criteria established by each respective healthcare providing entity.

In alternative exemplary arrangements, the exemplary method further comprises filtering the credential information of each of the plurality of healthcare providers to obtain healthcare providers with credential information matching the qualifying criteria established by a respective healthcare providing entity.

In alternative exemplary arrangements, the exemplary method further comprises receiving a healthcare providing entity request for release of credential information of healthcare providers matching the qualifying criteria established by the healthcare providing entity.

In alternative exemplary arrangements, the exemplary method further comprises communicating to a respective healthcare providing entity the credential information of healthcare providers matching the qualifying criteria established by the respective healthcare providing entity.

In alternative exemplary arrangements, the exemplary method further comprises implementing at least one security measure to safeguard the receipt, storage, and communication of credential information of each respective healthcare provider.

In alternative exemplary arrangements, the exemplary method further comprise autonomously verifying each of the plurality of healthcare provider's credential information by communicating with at least one outside source.

In alternative exemplary arrangements, the exemplary method further comprises sending a release authorization message to healthcare providers matching the qualifying criteria of a respective healthcare providing entity, wherein the release authorization message requires the healthcare provider to authorize or deny the communication of the healthcare provider's credential information to the healthcare providing entity.

Alternative exemplary arrangements provide a computer implemented method for centralizing the credentialing process of healthcare providers, comprising: receiving credential information from each of a plurality of healthcare providers; storing the respective credential information of each of the plurality of health care providers; autonomously verifying the authenticity of the credential information of each of the plurality of health care providers; storing qualifying criteria, wherein each of a plurality of healthcare providing entities includes respective qualifying criteria associated therewith, and wherein the qualifying criteria comprises specific credential information that the respective healthcare providing entity requires its health care providers to obtain; and communicating to each of the plurality of healthcare providing entities the credential information of a respective healthcare provider matching the qualifying criteria of the respective healthcare providing entity.

Alternative exemplary embodiments provide a computer implemented method for centralizing the credentialing process of healthcare providers, comprising: receiving credential information from each of a plurality of healthcare providers; storing the respective credential information of each of the plurality of healthcare providers; enabling a plurality of healthcare providing entities to establish qualifying criteria, wherein the qualifying criteria comprises specific credential information that the respective healthcare providing entity requires its healthcare providers to obtain; storing the respective qualifying criteria of each of the plurality of healthcare providers; filtering the credential information of each of the plurality of healthcare providers to obtain healthcare providers with credential information matching the qualifying criteria of a respective healthcare providing entity; and communicating to the respective healthcare providing entity the credential information of healthcare providers matching the qualifying criteria of the healthcare providing entity.

### BRIEF DESCRIPTIONS OF THE DRAWINGS

These and/or other aspects of the present general inventive concept will become apparent and more readily appreciated from the following description of the exemplary arrangements, taken in conjunction with the accompanying drawings of which:

FIG. 1 is an exemplary arrangement of an exemplary system;

FIG. 2 is an exemplary remote electronic device;

FIG. 3 is an exemplary method using the exemplary system; and

FIG. 4 is an alternative exemplary method using the exemplary system.

### DESCRIPTION OF INVENTION

Reference will now be made in detail to the exemplary arrangements of the present general inventive concept, examples of which are illustrated in the accompanying drawings, wherein like reference numerals refer to the like elements throughout. The exemplary embodiments are described below in order to explain the present general inventive concept by referring to the figures.

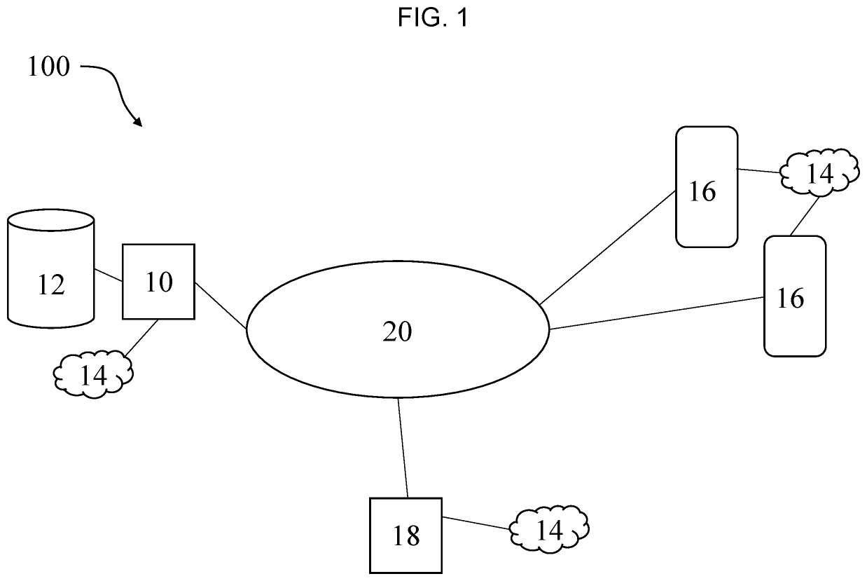

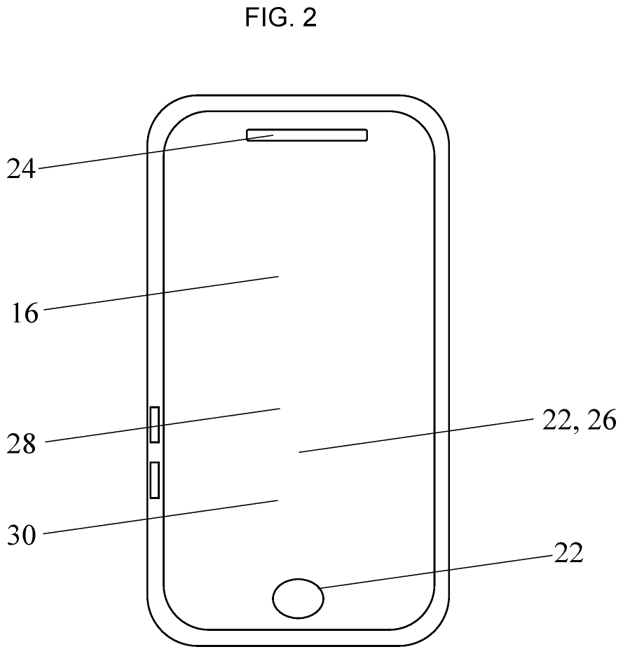

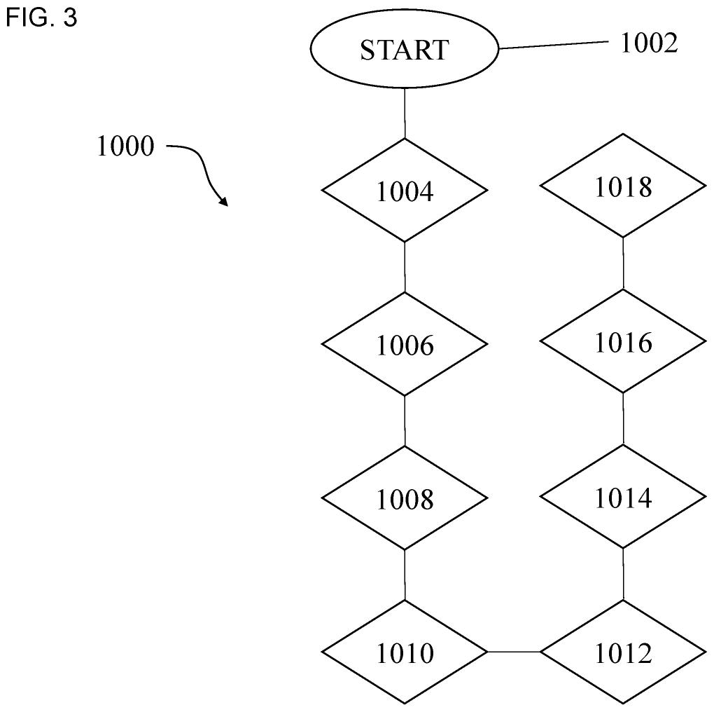

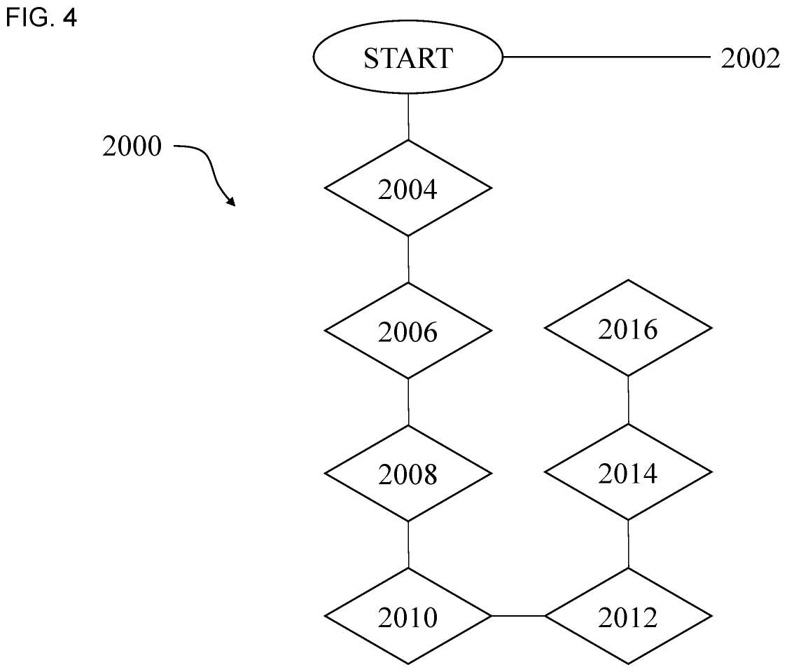

FIG. 1 is an exemplary arrangement of an exemplary system. FIG. 2 is an exemplary remote electronic device. FIG. 3 is an exemplary method using the exemplary system. FIG. 4 is an alternative exemplary method using the exemplary system.

The exemplary system and method for credentialing individual healthcare providers comprises a centralized platform for uploading necessary documents, data and sensitive information of individual healthcare providers via a secure system to facilitate credentialing of the individual healthcare provider with healthcare providing entities desiring or needing the individual healthcare provider's services. The exemplary system and method further provides a verification system designed to verify the individual healthcare provider's credential information, and documents to maintain integrity of the system and its users. However, the exemplary arrangements are not limited thereto.

The exemplary system and method provides a secure centralized credentialing platform that enables quick dissemination of an individual healthcare provider's credential documents, data and sensitive information to multiple healthcare providing entities while eliminating the need to store personal documents, data and sensitive information on personal devices, as well as eliminating the necessity of multiple transmissions of these documents, information and data through insecure channels.

Further, the exemplary system and method enables individual healthcare providers with the ability to request release or communication of their credential information, documents and data to specific healthcare providing entities. Additionally, the exemplary system and method enables healthcare providing entities that are registered with the system to request release or communication of individual healthcare providers' credential information, documents and data meeting certain qualifying criteria, and enabling the individual healthcare provider to authorize release of his/her respective information to the registered healthcare providing entity requesting release. However, the exemplary arrangements are not limited thereto.

Referring to FIGS. 1 through 4, the exemplary system and method for credentialing healthcare providers, designated generally as 100, is illustrated. The exemplary system comprises a central system server 10, a central system data store 12, system software 14, a plurality of remote electronic devices 16, at least one registered healthcare providing entity 18, and at least one communications network 20. In exemplary arrangements, the central system server 10, the plurality of remote electronic devices 16, and the at least one registered healthcare providing entity 18 are all configured to communicate via the at least one communications network 20 and through execution of the system software 14. Of course, this arrangement is merely exemplary, and in other embodiments, other arrangements may be used.

In exemplary arrangements, the exemplary central system server 10 includes one or more processors and data stores. The exemplary central system server 10 is configured to perform the various functions and operations herein described. The exemplary processors of the central system server 10 include at least one processor suitable for carrying out circuit executable instructions that are stored in the central system data store 12. The exemplary central system server 10 may include system circuitry located at a single location, or in a distributed arrangement that operates in a cloud environment, virtual server environment, or other suitable environment for performing the functions and operations describe herein. Numerous different types of system circuitry arrangements may be utilized in connection with the exemplary arrangements. As can be appreciated, this arrangement is merely exemplary, and in other embodiments, other arrangements may be used.

In exemplary arrangements, the exemplary central system data store 12 is in operative communication with the central system server 10. The exemplary central system data store 12 comprises one or a plurality of storage mediums. The exemplary storage mediums include one or more of several types of storage mediums suitable for holding or storing circuit executable instructions as well as other data. In exemplary arrangements, the exemplary central system data store 12 is configured to store circuit executable instructions that are executable by the central system server 10 as well as by processors associated with the plurality of remote electronic devices 16, as will be discussed. However, this arrangement is merely exemplary, and in other embodiments, other arrangements may be used.

The exemplary system software 14 comprises circuit executable instructions that are designed to be executed by the central system server 10 as well as processors associated with the plurality of remote electronic devices 16. The exemplary system software 14 includes a version that may be downloaded to a system user's remote electronic device 16. For example, the exemplary system software 14 may include a software application that is downloadable to a memory of a user's remote electronic device from app stores such as those of Apple®, Microsoft®, Android®, or similar. These arrangements are merely exemplary, and in other embodiments, other arrangements may be used.

In exemplary arrangements, the exemplary remote electronic device 16 includes at least one input device 22, at least one output device 24, a display screen 26, a processor 28, and a memory 30. The exemplary remote electronic device may comprise a smart phone, a laptop computer, a desktop computer, a smart pad, or other similar electronic device. This remote electronic device arrangement is exemplary, and in other embodiments, other electronic device arrangements may be used.

In exemplary arrangements, a user of the system is enabled to download a version of system software 14 that is configured to be executed by the processor 28 of the remote electronic device 16. The downloaded system software is stored in the memory 30 of the remote electronic device 16. In other exemplary arrangements, the system software 14 may be executed on, or downloaded from a website managed by the central system server 10, and that is accessible via the user's remote electronic device 16 through use of an internet browser. In still other exemplary arrangements, the system user may access the system software 14 by installing a copy on the memory 30 of the user's remote electronic device 16.

The exemplary processor 28 of the remote electronic device 16 is configured to execute the circuit executable instructions of the system software 14 to display a user interface 32 of the system software 14 on the display screen 26. The system user is thereafter enabled to interact with the system software 14 via inputs through the at least one input device 22 and outputs through the at least one output device 24 and/or the display screen 26. As can be appreciated, this arrangement is merely exemplary, and in other embodiments, other arrangements may be used.

In exemplary arrangements, the exemplary central system server 10 is operative to maintain a database of healthcare providing entities that are registered with the central system server 10. The at least one registered healthcare providing entity 18 comprises a healthcare providing entity that generally employs or contracts for the services of individual healthcare providers, and which typically performs credentialing or other verification of the individual healthcare provider before services begin. For example, the at least one registered healthcare providing entity 18 may comprise a nurse staffing company, a hospital, or other healthcare providing entity. Of course, the exemplary arrangements described herein may be used in other industries as well.

In exemplary arrangements, the central system server 10 is operative to communicate with the central system data store 12, the plurality of remote electronic devices 16, and the at least one registered healthcare providing entity 18 through at least one communications network 20. In exemplary arrangements, the communications between the devices, servers, registered entities, and data stores include at least one security measure. Exemplary security measures include encryption of transmitted data, use of VPNs, firewalls, antivirus software, as well as any other data and internet communications, wireless communications, and telecommunications protection methods. In addition, the exemplary system software 14 may also include any known security measure for protecting data and/or other information that is stored in a storage medium, such as the central system data store 12 and the memory 30 of an electronic device 16. These arrangements are merely exemplary, and in other embodiments, other arrangements may be used.

Through the exemplary user interface 32, the system user is enabled to upload his/her personal credential information, documentation, and data to the central system server 10. As used herein, credential information includes a system user's education information, years of experience, certifications and licenses, degrees, and any other information that may be associated with or required for credentialing of an individual healthcare provider. The system software 14, through the user interface 32, may prompt the user to upload his/her credential information to the central system 10. The exemplary system 10, after the system user has uploaded his/her credential information, is operative to cause the system user's uploaded personal credential information, documentation and data to be stored in the central system data store 12. For example, a prospective nurse looking for employment with a particular registered healthcare providing entity 18 will upload from the memory of the nurse's respective remote electronic device 16 or other internal or external memory device all of his/her credential information necessary for the desired registered healthcare providing entity 18 to appropriately credential the nurse for employment with the entity 18.

In some exemplary arrangements, the exemplary central system server 10 is operative to develop and maintain in the central system data store 12, a list of necessary credential information required for each registered healthcare providing entity. That is, the exemplary system 10 is configured to enable a healthcare providing entity 18 to provide a list of specific credential information that the healthcare providing entity 18 requires of their employed or contracted healthcare providers, before the healthcare providing entity 18 is willing to credential or verify the credential information of the healthcare providers. In this way, the individual healthcare provider is made aware through the user interface 32 of all the necessary credential information that a specific entity 18 must review in order for the individual healthcare provider to be credentialed by the entity 18. Of course, this arrangement is merely exemplary, and in other embodiments, other arrangements may be used.

The exemplary central system server 10 is operative to generate and associate a unique user identification code or other object with the respective system user such that the central system server 10 is aware that this particular system user is associated with the user's respective credential information. However, this arrangement is merely exemplary, and in other embodiments, other arrangements may be used.

In exemplary arrangements, the exemplary central system server 10 is configured to verify that the credential information uploaded by the system user is authenticate or truthful. In exemplary arrangements, the central system server 10 is operative to communicate with outside sources, for example, educational institutions, certifying or licensing agencies/entities or investigative background check entities to confirm that the credential information uploaded by a system user is authentic and truthful. Of course, these arrangements are merely exemplary, and in other embodiments, other arrangements may be used.

In alternative exemplary arrangements, exemplary system 10 is operative to automatically and autonomously communicate with outside sources to authenticate or confirm the truth of the individual healthcare provider's credential information. In this way, manual authentication of the credential information is avoided, thereby reducing the risk of human error. In such arrangements, exemplary system 10 may require the healthcare provider/user to provide the system 10 with authority to view or access the healthcare provider/user's credential information maintained at various outside sources. For example, the exemplary system 10 may integrate a platform such as Plaid®, which uses account holders' usernames and passwords to gain access to certain systems. Of course, this arrangement is merely exemplary, and in other embodiments, other arrangements may be used.

Once the system user's credential information is stored in the data store 12, the system 10 is operative to prompt the user through the user interface 32 to select at least one registered healthcare providing entity 18 to which the system user desires to transmit his/her respective credential information, or to which entity 18 the system user desires to grant access to review his/her credential information. Of course, this arrangement is merely exemplary, and in other embodiments, other arrangements may be used.

After the system user has selected at least one registered healthcare providing entity 18 for transmission of his/her credential information, the central system server 10 is operative to cause transmission of the system user's respective credential information to the selected registered healthcare providing entity 18. The system user's credential information is transmitted to the registered healthcare providing entity 18 through a secure channel or method of communication including at least one security measure. In some exemplary arrangements, the exemplary system user may receive a notification via the user's electronic device 16 from the central system server 10 that the entity 18 has received the system user's credential information. As can be appreciated, this arrangement is merely exemplary, and in other embodiments, other arrangements may be used.

In alternative exemplary arrangements, the selected registered healthcare providing entity is granted access to view the system user's credential information via the system software 14. In such arrangements, no transmission of credential information may take place, and the entity 18 is only enabled to view the credential information through the secure system. The system user may receive a notification via the user interface 32 that the entity 18 has accessed and viewed the system user's credential information.

In alternative exemplary arrangements, a registered healthcare providing entity 18 may request release of or access to the credential information of individual healthcare providers meeting certain qualifying criteria selected by the registered healthcare providing entity 18. In exemplary arrangements, the central system server 10 enables the registered healthcare providing entity 18 to select via the user interface 32 qualifying criteria such as educational information like a range of grade point average, type of institution, type of degree, certifications, licenses, as well as years of experience and/or other information or data that the registered healthcare providing entity deems necessary for potential employment candidates.

In such arrangements, the exemplary central system server 10 is configured to filter the system users based on system users whose credential information meets the qualifying criteria established by the registered healthcare providing entity 18. Thereafter, the central system server 10 is configured to only transmit or permit access to the credential information of system users meeting the qualifying criteria selected by the registered healthcare providing entity 18. Of course, this arrangement is merely exemplary, and in other embodiments, other arrangements may be used.

In arrangements in which the registered healthcare providing entity 18 establishes qualifying criteria, the exemplary central system server 10 may issue a notification to a system user meeting the qualifying criteria notifying the system user that they meet the criteria of a particular registered healthcare providing entity 18. The system user is thereafter enabled to authorize release of or grant access to the system user's credential information to the registered healthcare providing entity 18. In such arrangements, the exemplary central system server 10 may issue a second notification to the system user requesting authorization for the system 10 to transmit the system user's credential information to the registered healthcare providing entity 18.

In still other alternative exemplary arrangements, the central server system 10 may provide the system user with the option to select system profile preferences via the user interface 32. Such preferences may include enabling a system user to select a user preference in which the system user authorizes the central system server 10 to automatically transmit, or permit access to the system user's credential information to any requesting registered healthcare providing entity 18, or to any entity 18 that the system user meets the qualifying criteria thereof. Of course, these alternative arrangements are merely exemplary, and in other embodiments, other arrangements may be used.

In exemplary arrangements, once the registered healthcare providing entity 18 has made a determination whether to credential the respective system user, the central system server 10 is configured to facilitate further communication between the system user and the registered healthcare provider entity 18 by establishing a channel of communication between the system user and the registered healthcare providing entity 18. For example, the central system server 10 may establish a text messaging or chat room communication between the system user and the registered healthcare provider entity 18, or the central system server 10 may cause a disclosure of a telephone number at which the system user may be contacted and a telephone number at which the registered healthcare providing entity 18 may be contacted. In still other arrangements, the central system server 10 may cause notification messages to be sent between the system user and the registered healthcare providing entity 18 disclosing or requesting further information or data to be communicated through the system 10 between the parties. In this way, the system user and the entity 18 are enabled to continue discussion for employment or contracted services. Of course, this arrangement is merely exemplary, and in other embodiments, other arrangements may be used.

An exemplary method 1000 employing the exemplary system 100 is shown in FIG. 3. The exemplary method 1000 begins at 1002. At step 1004, a system user accesses the system software 14 via the system user's remote electronic device 16. At step 1006, the system user uploads their credential information to the central server system 10 via the user's remote electronic device. At step 1008, the central system server 10 is operative to verify the authenticity and truthfulness of the system user's uploaded credential information. At step 1010, the central system server 10 is operative to store the system user's uploaded credential information along with an associated user identifier in the central system data store 12. At step 1012, the central system server 10 prompts the system user to select at least one registered healthcare providing entity 18 to which the user desires to have his/her credential information and data transmitted, or to be made accessible. At step 1014, the central system server 10 is operative to transmit or make accessible the system user's respective credential information to the selected registered healthcare providing entity 18 utilizing at least one security measure. At step 1016, the central system server 10 is operative to cause at least one notification message to be sent to the user's remote electronic device 16 confirming that the registered healthcare providing entity 18 has received or accessed the system user's credential information. At step 1018, the central system server 10 is operative to cause further communication between the system user and the registered healthcare providing entity 18. As can be appreciated, this method is merely exemplary, and in other embodiments, other method steps or other orders of these steps may be taken.

An alternative exemplary method 2000 is shown in FIG. 4. The alternative exemplary method 2000 begins at step 2002. At step 2004, the registered healthcare providing entity 18 establishes qualifying criteria through the central system server 10. At step 2006, the registered healthcare providing entity 18 requests release of individual healthcare providers' credential information meeting the qualifying criteria. At step 2008, the central system server 10 filters the credential information of all system users to find individual healthcare providers whose credential information matches the established qualifying criteria of the entity 18. At step 2010, the central system server 10 sends at least one message to system users whose credential information matches the established qualifying criteria of the entity 18 requesting release of the system user's credential information. At step 2012, the system users may authorize or deny the release of, or access to his/her credential information to the requesting registered healthcare providing entity 18 via the user interface 32. At step 2014, the central system server 10 either transmits or makes accessible the credential information of the system users who have authorized release of their credential information to the requesting healthcare providing entity 18. At step 2016, the central system server 10 is operative to cause further communication between the registered healthcare providing entity 18 and the system user who has authorized release of his/her credential information to the entity 18. As can be appreciated, this method is merely exemplary, and in other embodiments, other method steps or other orders of these steps may be taken.

Although a few exemplary arrangements have been illustrated and described, it will be appreciated by those skilled in the art that changes may be made in these exemplary arrangements and methods, and other combinations of the features and relationships and other method steps and other orders of the method steps may be made to create new arrangements or methods without departing from the principles and spirit of the disclosure, the scope of which is defined in the appended claims and their equivalents.

What is claimed is:

1. A computer implemented method for centralizing the credentialing process of healthcare providers, comprising:
(a) receiving credential information from each of a plurality of healthcare providers;
(b) storing the respective credential information of each healthcare provider;
(c) verifying the authenticity of the respective credential information of each healthcare provider; and
(d) communicating credential information to at least one of a plurality of healthcare providing entities.

2. The computer implemented method according to claim 1, further comprising:
prior to (d), receiving from a respective healthcare provider a request to release the health care provider's credential information to at least one healthcare provider selected healthcare providing entity.

3. The computer implemented method according to claim 2, further comprising:
prior to (d), enabling a healthcare providing entity to establish qualifying criteria, wherein the qualifying criteria comprises specific credential information that the respective healthcare providing entity requires its healthcare providers to obtain.

4. The computer implemented method according to claim 3, further comprising:
prior to (d), storing the qualifying criteria established by each respective healthcare providing entity.

5. The computer implemented method according to claim 4, further comprising:
prior to (d), filtering the credential information of each of the plurality of healthcare providers to obtain healthcare providers with credential information matching the qualifying criteria established by a respective healthcare providing entity.

6. The computer implemented method according to claim 5, further comprising:
prior to (d), receiving a healthcare providing entity request for release of credential information of healthcare providers matching the qualifying criteria established by the healthcare providing entity.

7. The computer implemented method according to claim 6, further comprising:
wherein in (d), communicating to a respective healthcare providing entity the credential information of healthcare providers matching the qualifying criteria established by the respective healthcare providing entity.

8. The computer implemented method according to claim 7, further comprising:
prior to (a), implementing at least one security measure to safeguard the receipt, storage, and communication of credential information of each respective healthcare provider.

9. The computer implemented method according to claim 8, further comprising:
wherein in (c), autonomously verifying each of the plurality of healthcare provider's credential information by communicating with at least one outside source.

10. The computer implemented method according to claim 9, further comprising:
prior to (d), sending a release authorization message to healthcare providers matching the qualifying criteria of a respective healthcare providing entity, wherein the release authorization message requires the healthcare provider to authorize or deny the communication of the healthcare provider's credential information to the healthcare providing entity.

11. A computer implemented method for centralizing the credentialing process of healthcare providers, comprising:
(a) receiving credential information from each of a plurality of healthcare providers;
(b) storing the respective credential information of each of the plurality of health care providers;
(c) autonomously verifying the authenticity of the credential information of each of the plurality of health care providers;
(d) storing qualifying criteria, wherein each of a plurality of healthcare providing entities includes respective qualifying criteria in association therewith, and wherein the qualifying criteria comprises specific credential information that the respective healthcare providing entity requires its health care providers to obtain; and
(e) communicating to each of the plurality of healthcare providing entities the credential information of a respective healthcare provider matching the qualifying criteria of the respective healthcare providing entity.

12. The computer implemented method according to claim 11, further comprising:
prior to (d) enabling a healthcare providing entity to establish its respective qualifying criteria.

13. The computer implemented method according to claim 12, further comprising:
prior to (d), filtering the credential information of each of the plurality of healthcare providers to obtain healthcare providers with credential information matching the qualifying criteria associated with a respective healthcare providing entity.

14. The computer implemented method according to claim 13, further comprising:
prior to (d), receiving a healthcare providing entity request for release of credential information of healthcare providers matching the qualifying criteria established by the healthcare providing entity.

15. The computer implemented method according to claim 14, further comprising:
prior to (d), sending a release authorization message to healthcare providers matching the qualifying criteria of a respective healthcare providing entity, wherein the release authorization message requires the healthcare provider to authorize or deny communication of the healthcare provider's credential information to the healthcare providing entity.

16. The computer implemented method according to claim 15, further comprising:
wherein in (c), the credential information of each of the plurality of healthcare providers is verified by communication with at least one outside source.

17. A computer implemented method for centralizing the credentialing process of healthcare providers, comprising:
(a) receiving credential information from each of a plurality of healthcare providers;
(b) storing the respective credential information of each of the plurality of healthcare providers;
(c) enabling a plurality of healthcare providing entities to establish qualifying criteria, wherein the qualifying criteria comprises specific credential information that the respective healthcare providing entity requires its healthcare providers to obtain;
(d) storing the respective qualifying criteria of each of the plurality of healthcare providers;
(e) filtering the credential information of each of the plurality of healthcare providers to obtain healthcare providers with credential information matching the qualifying criteria of a respective healthcare providing entity; and
(f) communicating to the respective healthcare providing entity the credential information of healthcare providers matching the qualifying criteria of the healthcare providing entity.

18. The computer implemented method according to claim 17, further comprising:
prior to (f), autonomously verifying each of the healthcare provider's credential information by communicating with at least one outside source.

19. The computer implemented method according to claim 18, further comprising:
prior to (f), receiving a healthcare providing entity request for release of credential information associated with healthcare providers matching the qualifying criteria established by the healthcare providing entity.

20. The computer implemented method according to claim 19, further comprising:
prior to (f), sending a release authorization message to healthcare providers matching the qualifying criteria of a respective healthcare providing entity, wherein the release authorization message requires the healthcare provider to authorize or deny the communication of the healthcare provider's respective credential information to the respective healthcare providing entity.
